(19) United States
(12) Patent Application Publication
(10) Pub. No.: US 2008/0129961 A1
(43) Pub. Date: Jun. 5, 2008

(54) METHOD AND APPARATUS FOR MEASURING QUANTITY OF A FLUOROCHROME IN A BIOLOGICAL ENVIRONMENT

(76) Inventors: Abraham Schwartz, San Juan, PR (US); Rui Manuel Dias Cortesao dos Santos Bernardes, Sao Martinho do Bispo (PT); Stuart Francke, York, ME (US); Antonio Miguel Lino Santos Morgado, Casal da Misarela (PT)

Correspondence Address: WOOD, HERRON & EVANS, LLP, 2700 CAREW TOWER, 441 VINE STREET, CINCINNATI, OH, 45202, US

(21) Appl. No.: 11/946,993

(22) Filed: Nov. 29, 2007

Related U.S. Application Data
(60) Provisional application No. 60/867,933, filed on Nov. 30, 2006.

(51) Int. Cl. A61B 3/10; G01N 37/00

(52) U.S. Cl. 351/221; 351/205; 436/8

(57) ABSTRACT

A device containing one or more aqueous solutions comprising a fluorochrome in varying concentrations is used to quantitatively measure the fluorescence data collected from one or more tissues. Examples for making and using such a device during angiography to quantitatively measure the fluorescence data collected from a patient's eye is provided.

The present application claims the filing benefit of U.S. Provisional Application Ser. No. 60/867,933, filed Nov. 30, 2006, the disclosure of which is incorporated herein by reference in its entirety.

### FIELD OF THE INVENTION

The present invention relates to a system and method for quantitative analysis of optical imaging data, particularly fluorescence imaging such as that generated via angiography, by providing for standardization of fluorescence data.

### BACKGROUND OF THE INVENTION

Vascular and degenerative disorders of the retina and posterior segment of the eye include age-related macular degeneration (AMD), diabetic retinopathy, glaucoma, hypertensive retinopathy and vascular occlusions.

AMD is a common eye disease causing deterioration of the macula, the central area of the retina, a paper-thin tissue at the back of the eye where light-sensitive cells send visual signals to the brain. Sharp, clear “straight ahead” vision is processed by the macula. Damage to the macula results in the development of blind spots and blurred or distorted vision.

There are two types of AMD, usually referred to as “wet” and “dry” AMD. Dry AMD is the most common form, developing slowly and causing gradual loss of central vision. Wet AMD results from new blood vessels growing behind the retina, causing bleeding, scarring and loss of sight. Wet AMD can develop quickly but may be responsive to treatment in the early stages. AMD usually affects individuals older than 50 years of age and is the major cause of visual impairment in the United States.

For nearly four decades, ophthalmologists have relied on image-based techniques, such as fluorescein angiography which utilizes a specialized fundus camera to capture rapid-sequence photographs of the retinal vasculature following an intravenous injection of a fluorescent reagent, for example, fluorescein or indocyanin green. Image-based techniques have also relied on not only exogenous fluorophores, but also, in vivo autofluorescence. Currently, fluorescein angiography is an important tool for clinical diagnosis and for the develop and monitoring of a treatment plan for retinal disorders, including, but not limited to, AMD, diabetic retinopathy, hypertensive retinopathy and vascular occlusions.

Even though fluorescein angiography facilitates in vivo observation of retinal circulation and is used for management of AMD and other retinal disorders, currently fluorescein angiography provides only a subjective image-based diagnostic tool and has not been used to provide quantitative data because there is no fluorescent standard available to calibrate ophthalmaloscopic instruments. There is a need for a device and method useful to reliably and quantitatively measure fluorescence during angiography to effectively diagnose and monitor treatment of vascular and degenerative disorders of the retina and posterior segment of the eye.

### SUMMARY OF THE INVENTION

In one aspect the invention comprises one or more fluorescence standard solutions of a fluorochrome providing substantially the same spectral and intensity properties as the fluorochrome in the tissues of the eye (serum, vitreous humor, corneal tissue, etc.). In another aspect, the invention comprises a device for containing one or more fluorescence standard solutions. In another aspect, the invention comprises the use of one or more fluorescence standard solutions to quantitatively measure the fluorescence of at least one or more tissues of the eye by comparing the fluorescence data collected from the eye with the fluorescence data of the standard solution(s).

Unless otherwise defined herein, scientific and technical terms used in connection with the present invention have the meanings commonly understood by those of ordinary skill in the art. Further, unless otherwise required by context, singular terms include pluralities and plural terms include singular.

The term “focal axis” as used herein refers to an axis originating at the eye and extending toward an object of intended visual recognition.

The term “fluorochrome” as used herein refers to any fluorescing compound endogenous or exogenous to the eye. Examples of endogenous and exogenous fluorochromes are described below.

The term “fluorescence behavior” as used herein refers to a phenomenon whereby the fluorescence emitted by a given fluorochrome is determined by the environment in which the fluorochrome is present (e.g. quantum efficiency).

The term “fluorescence data” as used herein refers to fluorescence emissions from the eye and/or fluorescence standard solution. Such data includes, but is not limited to, fluorescence images of the eye and/or standard solution contained within a containment device.

The abbreviations used herein have their usual meaning in the art. For clarity, the meanings of certain abbreviations are as follows: “AMD” means age-related macular degeneration; “ICG” means indocyanin green; “SLO” means scanning laser ophthalmoscope; “PBS” means phosphate buffered saline; “BSA” means bovine serum albumin; and “HA” means hyaluronic acid.

The above and other objects and advantages of the present invention shall be made apparent from the accompanying drawings and the description thereof.

### BRIEF DESCRIPTION OF THE DRAWINGS

The accompanying drawings, which are incorporated in and constitute a part of this specification, illustrate embodiments of the invention and, together with a general description of the invention provided above, and the detailed description of the embodiments provided below, serve to explain the principles of the invention.

FIG. 1 is a view of the upper surface of a standard cell according to one embodiment of the invention.

FIG. 2 is a view of the upper surface of a standard cell according to another embodiment of the invention.

FIG. 3 is a schematic diagram showing a system for capturing fluorescence data according to one embodiment of the present invention.

FIG. 4 is a schematic diagram showing a system for capturing fluorescence data according to another embodiment of the invention.

FIG. 5 is a schematic diagram showing a system for capturing fluorescence data according to yet another embodiment of the invention.

FIG. 6 is a schematic drawing showing an alternative adaptor for capturing fluorescence data from a standard cell.

FIG. 7 is an isometric cross-section of the adaptor of FIG. 6.

FIG. 8 is an example of a calibration plot showing the concentration of fluorescein (ng/ml) is serum vs. instrument reading.

### DETAILED DESCRIPTION OF THE INVENTION

### Fluorescence Standard Solutions

Fluorescence standard solutions are prepared using any fluorochrome that allows the benefits of the present invention to be achieved. The fluorochrome can be naturally occurring or synthetic and can be endogenous or exogenous to the eye of the subject. Examples of endogenous fluorochromes are known in the art and include, without limitation, corneal fluorophores, crystalline lens fluorophores, vitreous fluorophores, lipofuscin and melanin. Endogenous fluorochromes are described by F. Docchio in Introduction to Ocular Fluorometry, chapter 4 “A review of endogenous ocular fluorophores” (European Concerted Action on Ocular Fluorometry—EUROEYE, 1997), incorporated herein in entirety. Exogenous fluorochromes include, without limitation, fluorescein free acid (“fluorescein”), fluorescein glucuronide, ICG and hematoporphyrin and derivatives thereof. Exogenous fluorophores are described by F. Docchio in Introduction to Ocular Fluorometry, chapter 5 “A review of exogenous ocular fluorophores” (European Concerted Acion on Ocular Flurometry—EUROEYE, 1997), incorporated herein in entirety.

In order to determine the suitability of a fluorochrome for use as a standard in fluorescence angiography, it is recommended to determine the reduction of fluorescence intensity of the selected fluorochrome caused by biological fluids. To do this, a selected fluorochrome is dissolved in an aqueous solution to make a first set of standards. Examples of aqueous solutions useful for dissolving the fluorochrome include, without limitation, deionized water, saline, or a buffered saline solution such as PBS, TRIS-buffered saline or any other buffer compatible with the purpose of the present invention.

A second set of standard solutions are prepared in a relevant biological fluid including, without limitation, serum or vitreous humor so that reduction of fluorescence intensity of the selected fluorochrome in the biological fluid can be determined. The same fluorochrome used to make the first set of standard solutions is added to serum, for example, human serum, or vitreous humor, for example, human vitreous humor.

The first and second sets of standard solutions are used to determine the reduction of fluorescence intensity for a particular fluorochrome caused by an environment comprising a biologic fluid. The reduction of fluorescence intensity for a particular fluorochrome is determined by first measuring the fluorescence intensities of the selected fluorochrome in the first set of standard solutions (those prepared in aqueous solution) and comparing the fluorescence intensities to fluorescence intensities of the fluorochrome in the second set of standard solutions (those prepared in biological fluids). For example, the fluorescence intensity of a fluorochrome at one or more concentrations in PBS at pH 7.4 is measured and compared to the fluorescence intensity of the same fluorochrome at the same concentrations in serum and/or vitreous humor and/or other biological fluid. Fluorescence intensities are measured and compared using an appropriate instrument, for example, a spectrofluorometer such as a Spex Fluoromax™ (Horiba Jobin Yvon, Edison, N.J.). A standard curve or linear plot of the fluorescence intensity measurements of each of a set of concentrations in a first set of standard solutions (in aqueous solution) is established and compared to a standard curve or linear plot of fluorescence intensity measurements of each of a set of concentrations in a second set of standard solutions (in biological fluid) to determine the degree of increase or reduction of fluorescence intensity of a given fluorochrome. When the effect of a biological fluid environment on the reduction of fluorescence intensity for the fluorochrome of choice is determined, a working standard solution is prepared for use in connection with fluorescence angiography of patients.

A working set of standard solutions is prepared using a synthetic solution that mimics a biological fluid. For example, a commercially available synthetic human serum (Irvine Scientific, Santa Ana, Calif.) or 1% BSA in PBS at pH 7.4 is suitable. Also, PBS at pH 5.5 or hyaluronic acid (“HA”) in PBS is useful to mimic vitreous humor. For example, 0.1-5.0% HA, w/v, in PBS serves as a synthetic substitute for vitreous humor. Optionally, gelatin is added to the working standard solutions, for example, in the range of about 1-3% gelatin (w/v). The working standard solutions are contained within a standard cell device, described below, during use.

### Standard Cell

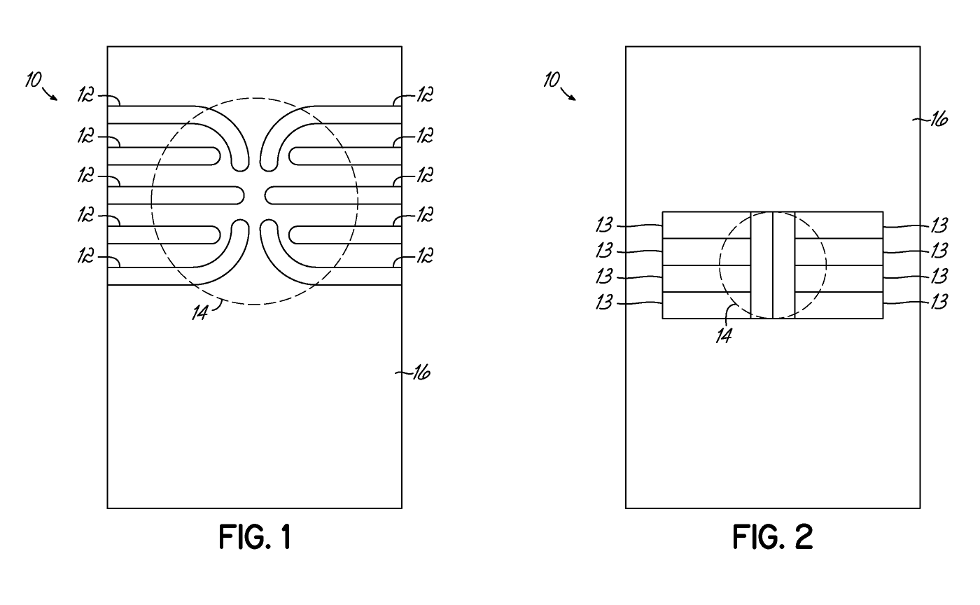

Referring to FIG. 1, a standard cell 10 is used to contain one or more working standard solutions in one or more channels 12. In the embodiment shown in FIG. 1, ten channels 12 are shown, but the cell 10 may comprise 1 to 100 or more channels 12 depending upon the size of the cell 10 and the channels 12. The channels 12 are arranged and shaped so as to be impacted by an energy source, such as light, directed at the cell 10. A dotted line indicates the circular area of impact 14. As shown in FIG. 1, some the channels 12 may be curved so as to be included within the area of impact 14. However, any alternative configuration of channels 12 is possible as long as a portion of each channel 12 is within the area of impact 14.

The standard cell 10 as shown in FIG. 1 is formed by any manner known in the art from a material that is substantially transparent to the spectral range of light used to excite the particular fluorochrome used. For example, a plate or slide 16, typically glass or plastic polymer, may be etched, milled or molded to form one or more channels 12. In use, a cover, such as a coverslip (not shown), may be placed over the channels 12 to seal the channels and contain the working standard solutions. The cover may be removably or permanently positioned on the standard cell 10. Also, the cover may be placed on the cell 10 prior to addition of the standard solutions with the channels 12 filling by capillary action or it may be positioned after the channels 12 are filled.

In another embodiment, shown in FIG. 2, a standard cell 10 comprises a plate or slide 16 having attached thereto one or more compartments 13 comprising capillary tubes or cuvettes attached to the plate or slide 16 by any method, including adhesive attachment or welding. In use, the channels 13 contain working standard solutions.

### System for Angiography

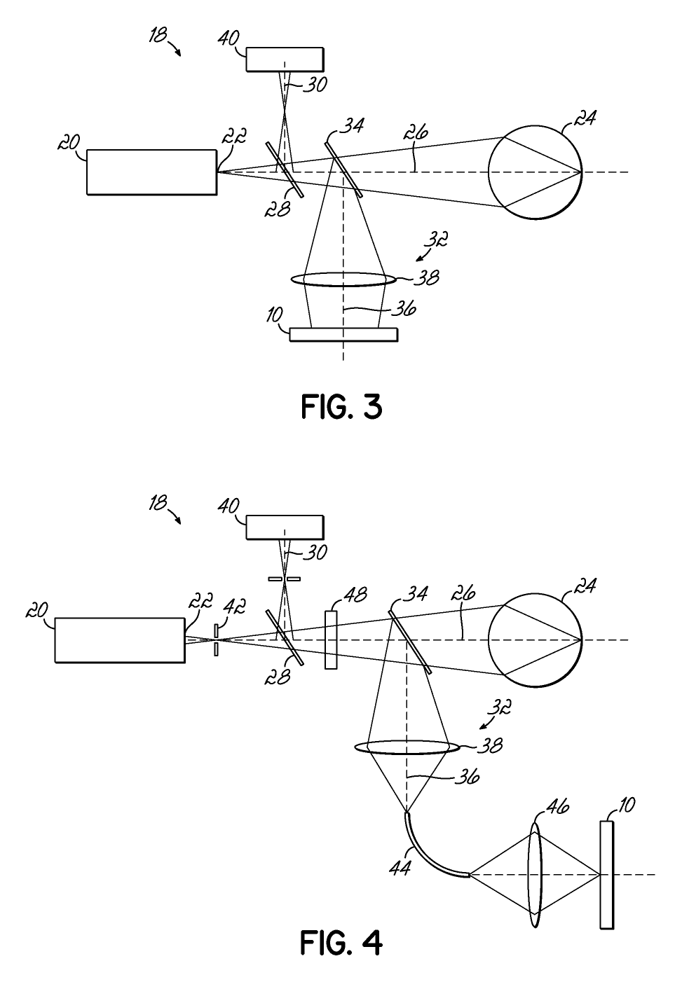

FIGS. 3-5 show various schematic drawings of systems useful for simultaneous collection of fluorescence data from the standard cell and a patient's eye during angiography as contemplated by the invention. Referring to FIG. 3, the system includes an instrument 18 that emits a beam of energy, such as a light beam, and collects fluorescence data; a common instrument for such use is a scanning laser ophthalmoscope (SLO) 18, known in the art, for example, a Heidelberg Retina Angiograph 2 (Heidelberg Engineering, Heidelberg, Germany). Such example is not intended to be limiting. The invention contemplates other SLO instruments and other instruments used for fluorescence measurements performed on biological fluids and is not limited to any particular instrument or even to angiography.

The instrument 18 contains a light source 20 for transmission of excitation light 22 to an eye 24 of a subject patient (injected intravenously with a clinical grade fluorochrome as is known in the art) over a focal axis 26. A dichroic mirror 28 reflects emission light from the eye 24 or from a standard cell 10 to a detector axis 30.

An adaptor apparatus 32 includes a beam splitter 34 for splitting the excitation light 22 between the focal axis 26 directed to the eye 24 and a standardization axis 36 directed to the standard cell 10. Excitation light 22 reflected along the standardization axis 36 passes through an adaptor lens 38 to focus the light onto the standard cell 10.

The excitation light 22 produces fluorescence emissions from the eye 24 and the standard cell 10. Emissions from the eye 24 are directed to a detector 40 within the SLO 18, or separately contained, by the dichroic mirror 28. Emissions from the standard cell 10 are directed to the detector 40 via both the beam splitter 34 of the adaptor apparatus 32 and the dichroic mirror 28 of the instrument 18. The detector 40 is used to quantitatively determine the amount of fluorescence emitted from the eye by comparison of the emissions from the eye 24 with emissions from standard solutions contained in the standard cell 10.

FIG. 4 is a schematic representation of a system similar to that of FIG. 3 but with additional features. For example, a pinhole device 42 may be provided along the focal axis 26 to facilitate focusing of the excitation light 22 on the eye 24. The pinhole device 42 is preferably positioned between the excitation light 22 and the dichroic mirror 28. Also shown in FIG. 4 is an optical fiber bundle 44 for receiving light directed along the standardization axis 36 to a second lens 46 for focusing light on a standard cell 10. Likewise, the optical fiber bundle 44 can direct emissions from the standard cell 10 to the beam splitter 34. Use of an optical fiber bundle 44 permits the standard cell 10 to be positioned in an area away from the patient, reducing the size of the apparatus situated near the patient and decreasing the risk of damage to the standard cell 10. Optionally, an XY scanner 48, as is known in the art, may be included in the system, shown in FIG.4 positioned between the dichroic mirroe 38 and the beam splitter 34.

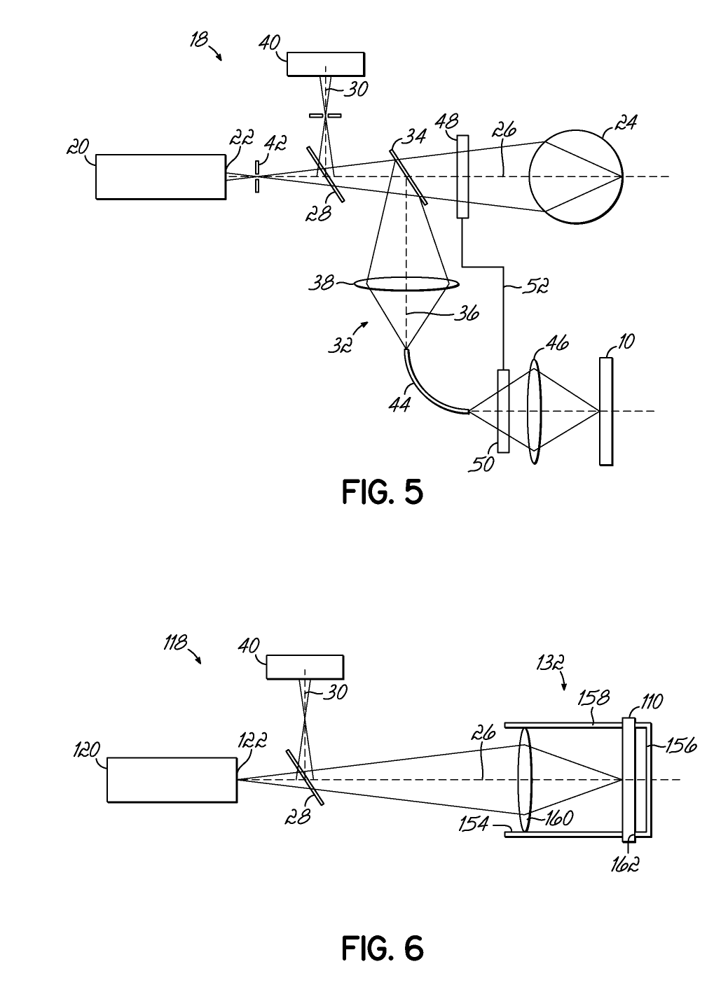

FIG. 5 show yet another embodiment in schematic form for simultaneous collection of fluorescence data during angiography. The features are as described above with several additional features. An XY scanner 48 as is known in the art may be placed downstream of the beam splitter 34 along the focal axis 26. An adaptor XY splitter 50 may be placed in the standardization axis 36. A synchronization connection 52 may connect the XY scanner 48 and the XY splitter 50 as is known in the art.

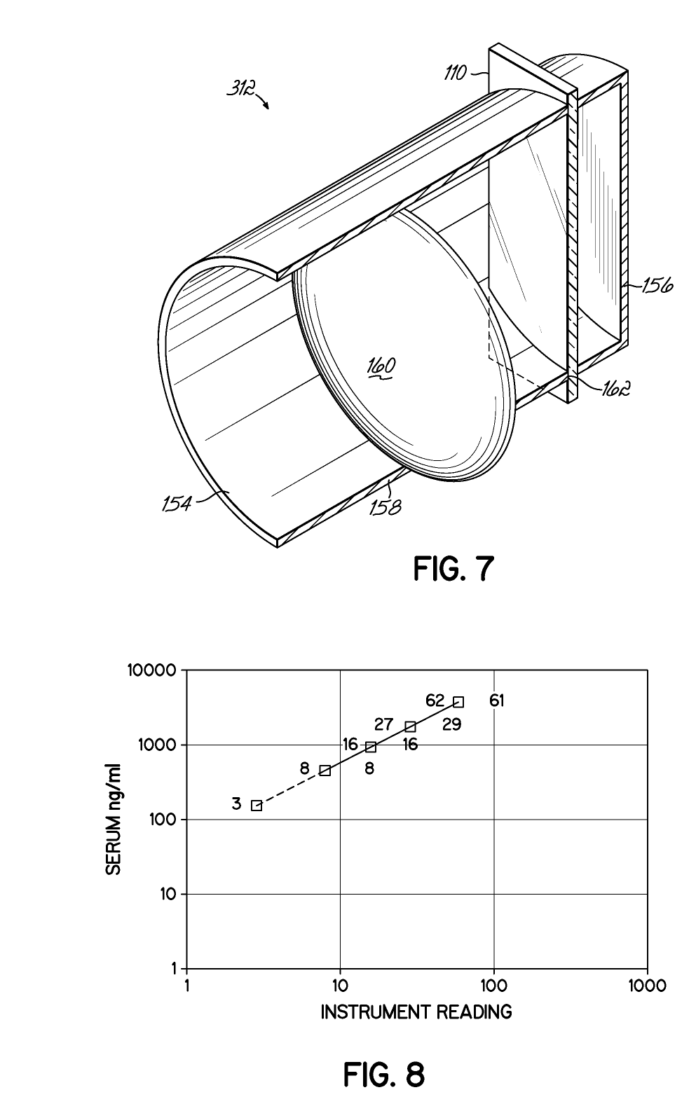

FIG. 6 is a schematic drawing of a system useful for measuring fluorescence data from a standard cell using the same instrument settings as those used to collect fluorescence data from a patient's eye. In this embodiment, an adaptor apparatus 132 is fitted over the eyepiece of the instrument 118, thereby replacing the patient's eye. As shown in FIG. 7, the adaptor apparatus 132 comprises an open end 154 for fitting over the eyepiece of the instrument 118 (not shown in FIG. 7), a closed end 156 and sidewall 158 enclosing the space between the open end 154 and closed end 156. In between the open end 154 and closed end 156 enclosed within the sidewall 158 is a lens 160 that approximates or mimics the lens of an eye. Between the lens 160 and closed end 156 is a slot 162 in the sidewall 158 for placement of a standard cell 110 perpendicular to the path of excitation light 122 transmitted by light source 120 the instrument 118, as shown in FIG. 6. The adaptor apparatus 132 containing a standard cell 110 is used after fluorescence image data is collected for a patient's eye wherein another set of fluorescence image data for the standard cell 110 is collected using the exact instrument settings as those used for the patient. The adaptor apparatus 132 may be made from metal, such as aluminum, or plastic polymers, or other suitable material capable of being shaped to fit over the eyepiece of the instrument 118.

### EXAMPLES

### Determination of Reduction of Fluorescence Intensity of Fluorescein caused by Biological Fluids

A fluorochrome, fluorescein, was selected as a candidate for use in the standardization system and methods of the present invention. To determine its suitability for such use, a set of standards was prepared by dissolving fluorescein (Acros Organics, Fair Lawn, N.J.) in PBS at pH 7.4 and 25° C. in each of the following concentrations expressed as nanograms (fluorescein) per ml (PBS): 0,100, 200, 400, 800. This set of standards is referred to as the “aqueous standards.”

Another set of standards was prepared by dissolving fluorescein in human vitreous humor at 25° C. in each of the following concentrations (ng/ml): 0, 100, 200, 400, 800. This set of standards is referred to as “vitreous humor standards.”

Another set of biological fluid standards was prepared by dissolving fluorescein in human serum at 25° C. in the following concentrations (ng/ml): 0, 300, 600, 1200, 2400. This set of standards is referred to as “serum standards.”

The fluorescence intensities of each of the aqueous standards described above were determined using a spectrofluorometer, as is commonly practiced in the art, and a plot of fluorescence intensity versus concentration was created. Fluorescence intensities of each of the vitreous humor standards and of each of the serum standards were likewise determined and fluorescence intensities versus concentration for each of the serum standard and vitreous humor standard were plotted.

Comparison of fluorescence intensities of the serum standards to the plot created for the aqueous (PBS) solutions showed no shift in spectral position or change in spectral shape. The reduction in fluorescence intensities of the serum standards was about 82% compared to the fluorescence intensities of the aqueous standards. Comparison of fluorescence intensities of the vitreous humor standards to the plot created for the aqueous solutions showed no shift in spectral position or change in spectral shape. The reduction in fluorescence intensities of the vitreous humor standards was about 5% compared to the fluorescence intensities of the aqueous standards.

When testing other fluorochromes, in general, the shift in spectral position or change in spectral shape should be no greater than about 10% and preferably, less than 5%. The reduction in fluorescence intensity is corrected by adjusting the concentration of the fluorochrome in the working solution.

As a further example, indocyanin green (ICG) was tested for suitability as fluorescence standard. ICG (Acros Organics, Fair Lawn, N.J.) was dissolved in PBS, pH 7.4, with 0.1% Tween 20 at 25° C. to make the aqueous standards. Fluorescence of a series of 1:2 dilutions were made with a spectrofluorometer beginning with a maximum concentration of 100 ng/ml down to a “blank” comprising PBS, pH 7.4, with 0.1% Tween with essentially no ICG (only trace amounts).

Since ICG is not completely soluble when mixed directly with serum or vitreous humor, ICG was first dissolved in PBS, pH 7.4, containing 0.1% Tween 20 at a higher concentration (≧1000 ng/ml) and then mixed with bovine vitreous humor for one set of biological fluid standards, or with human serum, for another set of biological fluid standards. When mixed in this way, ICG was observed to remain in solution in serum and in vitreous humor and to maintain its spectra and quantum yield as compared to ICG in PBS with 0.1% Tween 20. Further, the emission intensity of ICG was found to be stable across a pH range of 5-10.

### Preparation of Standard Cell with Working Standard Solutions

Due to variability and rather poor shelf life of biological fluids, sets of working standard solutions were prepared using either PBS at pH 5.5 to mimic human serum and/or PBS at pH 7.3 to mimic human vitreous humor. For example, fluorescein was dissolved in PBS at pH 5.6 in the following concentrations: 0, 300, 600, 1200 and 2400 ng/ml, to serve as working standard solutions to mimic human serum standards. Fluorescein was added to PBS at pH 7.3 containing 1.0% hyaluronic acid (HA) in the following concentrations: 0, 100, 200, 400 and 800 ng/ml, to serve as working standard solutions to mimic human vitreous humor. HA may be used in a range from about 0.1% to 5% (w/v) in PBS at pH 7.3 to mimic vitreous humor.

To prepare a standard cell, gelatin in a concentration of from 1-3% is added to the working solutions and heated to about 50° C. or other temperature sufficient to dissolve the gelatin, and the working solutions are loaded into the channels 12 or compartments 13 of a standard cell 10 via capillary action or other means, such that different known concentrations of fluorescein in working standard solution are contained in separate channels 12 or compartments 13. Allowing the gelatin to solidify prevents formation of bubbles in the working standard solutions in the standard cell 10. Optionally, after the working standards are loaded into the standard cell 10, the ends of the channels 12 or compartments 13 may be sealed, for example, with epoxy or other non-fluorescent sealant that does not leach into the working standard solutions.

### Acquiring Fluorescence Intensity Images from an Eye and from Working Standards

Use of a SLO instrument is according to manufacturer's instructions. Typically, as an example for use in simultaneous collection of data from a patient's eye and from working standards in a standard cell 10 as shown in FIGS. 3-5, the gain and focus of the SLO instrument 18 was adjusted to the retinal plane of the eye 24 such that the fluorescent image of the retina was optimal for digital fluorescence images in the range from image 23 through 30. The adaptor lens 38 was adjusted on the standard cell 10 such that the fluorescence data for the standard cell 10 comprised about digital images 3 through 8.

A subject patient is injected in an arm vein with clinical grade fluorescein (Novartis AG, Basel, Switzerland) and fluorescence data (images) collected approximately 10 minutes after i.v. injection. For example, about 32 digital fluorescence images were collected at a focal spacing of approximately 300 microns within about 2 seconds of time using a SLO instrument. The data from the eye 24 of the subject and the standard cell 10 was taken simultaneously. Alternatively, as shown in FIG. 6, after fluorescence data is collected from the eye, fluorescence data is then from the standard cell 110 as contained within an adaptor apparatus 132 fitted over the eyepiece of the SLO instrument using identical instrument settings for each.

### Analyzing Fluorescence Data from the Patient and Standards

In the example of simultaneous collection of data, optimal data from the working standards was generally about the 5^{th} image of digital data collected and optimal data from the patient was typically about the 23^{rd} image of digital data. The average intensity of each of the channels 12 or compartments 13 in the standard cell 10, whether containing working standards prepared from the serum mimic or from the vitreous humor mimic, was determined using an area 15×15 pixel in size. The values of the fluorescence measurements of the set of working standard solutions were plotted by linear regression in ng/ml and served as calibration plots. The blank standard is used to determine the detection threshold. Point locations or areas on the patient's optimal image were measured and intensities were determined by comparison against the appropriate calibration plot. For measurements made from point locations or areas of the patient's eye comprising blood vessels, comparisons were made to the calibration plot for the serum mimic working standard. For measurements from the “open area” (not within a blood vessel) of the eye, comparisons were made to the calibration plot for vitreous humor working standard solutions.

Calibration plots for the blood and vitreous humor compartments of the eye were determined by linear regression of the corresponding standard data. These calibration plots were used to calculate instrument performance parameters. An exemplary calibration plot is depicted in FIG. 8 for measurements taken within a blood vessel of a patient's eye and the serum-mimic working standard solutions. As is standard in the art, linear regression is calculated and plotted on a log-log graph to create a calibration plot. The data points are represented in FIG. 8 as solid dots and boxes are used to represent the regressed values on the calibration plot. A qualitative estimate of linearity of the instrument was determined by calculating the average residual percent; average residual percent is calculated by determining the percent each of the data points is not on the regressed line and then calculating their absolute average. Preferably, an algorithm contained within computer software is used to convert an instrument's intensity reading in ng/ml using the calibration regression plot.

### Producing a Quantitative Color Image

Optionally, fluorescence intensity data from a patient may be “translated” or converted to a color image record. An exemplary way to produce a color image is to assign specific colors to specific concentrations of fluorochrome as determined by the fluorescence intensity calibration plot generated from the set of working standards. For example, 0 ng/ml could be expressed as blue, 100 ng/ml as green, 200 ng/ml as yellow, etc. for the full range of the calibration plot, with all points in between represented by appropriate transitional colors. The instrument settings used to generate the patient's fluorescence intensity data must be the same as those used to generate the fluorescence intensity data collected from the working standards. The concentration represented by each pixel of a patient's fluorescence image is then “assigned” its corresponding color and a color image is constructed to represent a quantitative image of the tissue examined. Such quantitative color images provide an easy means for comparing images collected over time from the same patient or for comparing images collected from multiple subjects.

While specific embodiments of the subject invention have been discussed, the description included herein is intended to be illustrative and not limited. Variation of the invention will be apparent to those skilled in the art and the invention should be interpreted to include a full scope of equivalents.

What is claimed is:

1. A device comprising:
a. a first plate having one or more channels;
b. a calibration reagent comprising a fluorochrome in aqueous solution contained in said one or more channels; and
c. a second plate covering said one or more channels,
wherein at least the second plate, and optionally the first plate, is transparent to a spectral range of light capable of exciting the fluorochrome.

2. The device of claim 1 wherein at least one of said first and second transparent plates comprises glass, quartz, or a plastic polymer.

3. The device of claim 1 wherein said aqueous solution is phosphate buffered saline.

4. The device of claim 1 wherein said first plate has at least two channels and the aqueous solution in at least two channels contains a different concentration of the fluorochrome.

5. The device of claim 1 wherein said aqueous solution comprises phosphate buffered saline at pH 5.5 and optionally containing gelatin at a concentration of between 1-3% (w/v).

6. The device of claim 1 wherein said aqueous solution comprises phosphate buffered saline at pH 7.3 and further comprises hyaluronic acid at a concentration of between 0.1-5%.

7. The device of claim 1 wherein said fluorochrome is one of: fluorescein, fluorescein glucuronide, indocyanin green, lipofuscin, melanin, hematoporphyrin, corneal fluorophores, crystalline lens fluorophores and vitreous fluorophores.

8. A device comprising:
a. a plate having one or more capillary tubes attached to a surface of the plate; and
b. a calibration reagent comprising a fluorochrome in aqueous solution contained within said one or more capillary tubes,
wherein said one or more capillary tubes is transparent to a spectral range of light capable of exciting the fluorochrome.

9. The device of claim 8 wherein the plate comprises glass, quartz or plastic polymer having one or more capillary tubes adhesively attached to the surface of the plate.

10. The device of claim 8 wherein said aqueous solution is phosphate buffered saline.

11. The device of claim 8 having at least two capillary tubes attached to the surface wherein the reagent contained within each of said two capillary tubes comprises a different concentration of fluorochrome.

12. The device of claim 8 wherein said aqueous solution comprises phosphate buffered saline at pH 5.5 and optionally containing gelatin at a concentration of between 1-3% (w/v).

13. The device of claim 8 wherein said aqueous solution comprises phosphate buffered saline at pH 7.3 and further comprises hyaluronic acid at a concentration of between 0.1-5%.

14. A method for quantitative measurement of fluorescence in one or more tissues of a human eye comprising:
a. using an instrument capable of emitting a light wherein said light is divided by a beam splitter into a first beam directed into the eye and a second beam directed to the device of claim 1;
b. collecting fluorescence data from one or more tissues of the eye and from the device; and
c. determining the quantity of fluorochrome in said one or more tissues of the eye by comparing fluorescence data from one or more tissues of the eye with fluorescence data from the device.

15. The method of claim 14 wherein said one or more tissues is selected from: blood within blood vessels, vitreous humor, cornea, lens and retina.

16. A method for quantitative measurement of fluorescence in one or more tissues of a human eye comprising:
a. using an instrument capable of emitting a light wherein said light is directed into the eye and collecting fluorescence data from one or more tissues of the eye;
b. replacing the eye with the device of claim 1 and collecting fluorescence data from the fluorochrome contained with said device; and
c. determining the quantity of fluorochrome in said one or more tissues of the eye by comparing the fluorescence data from one or more tissues of the eye with fluorescence data from the device.

17. The method of claim 16 wherein said one or more tissues is selected from: blood within blood vessels, vitreous humor, cornea, lens and retina.

18. A method for quantitative measurement of fluorescence in one or more tissues of a human eye comprising:
a. using an instrument capable of emitting a light wherein said light is divided by a beam splitter into a first beam directed into the eye and a second beam directed to the device of claim 8;
b. collecting fluorescence data from one or more tissues of the eye and from the device; and
c. determining the quantity of fluorochrome in said one or more tissues of the eye by comparing fluorescence data from one or more tissues of the eye with fluorescence data from the device.

19. The method of claim 18 wherein said one or more tissues is selected from: blood within blood vessels, vitreous humor, cornea, lens and retina.

20. A method for quantitative measurement of fluorescence in one or more tissues of a human eye comprising:
a. using an instrument capable of emitting a light wherein said light is directed into the eye and collecting fluorescence data from one or more tissues of the eye;
b. replacing the eye with the device of claim 8 and collecting fluorescence data from the fluorochrome contained with said device; and
c. determining the quantity of fluorochrome in said one or more tissues of the eye by comparing the fluorescence data from one or more tissues of the eye with fluorescence data from the device.

21. The method of claim 20 wherein said one or more tissues is selected from: blood within blood vessels, vitreous humor, cornea, lens and retina.

22. A method of preparing standard reagent comprising:
a. preparing a first set of solutions by dissolving a fluorochrome at each of two or more concentrations in phosphate buffered saline pH 7.4;
b. preparing a second set of solutions by dissolving the fluorochrome at each of two or more concentrations in serum;
c. measuring fluorescence intensities of the first set of solutions and preparing a first graph of fluorescence intensities versus concentration;
d. measuring fluorescence intensities of the second set of solutions and preparing a second graph of fluorescence intensities versus concentration;
e. comparing the first graph and second graph for shift in spectral position, change in spectral shape and change in fluorescence intensities; and
f. preparing a third set of solutions by dissolving the fluorochrome at each of two or more concentration in phosphate buffered saline at about pH 5.5 only if the shift in spectral position and change in spectral shape of the second set of solutions compared to the first set of solutions are less than 10%.

23. A method of preparing standard reagent comprising:
a. preparing a first set of solutions by dissolving a fluorochrome at each of two or more concentrations in phosphate buffered saline pH 7.4;
b. preparing a second set of solutions by dissolving the fluorochrome at each of two or more concentrations in vitreous humor;
c. measuring fluorescence intensities of the first set of solutions and preparing a first graph of fluorescence intensities versus concentration;
d. measuring fluorescence intensities of the second set of solutions and preparing a second graph of fluorescence intensities versus concentration;
e. comparing the first graph and second graph for shift in spectral position, change in spectral shape and change in fluorescence intensities; and
f. preparing a third set of solutions by dissolving the fluorochrome at each of two or more concentration in phosphate buffered saline at about pH 7.3 containing hyaluronic acid in a range from about 0.1%-5.0% (w/v) only if the shift in spectral position and change in spectral shape of the second set of solutions compared to the first set of solutions are less than 10%.

24. The method of claim 22 wherein gelatin in a concentration of about 1-3% (w/v) is added to the third set of solutions.

25. The method of claim 23 wherein gelatin in a concentration of about 1-3% (w/v) is added to the third set of solutions.
